(19) United States
(12) Patent Application Publication
(10) Pub. No.: US 2013/0102932 A1
(43) Pub. Date: Apr. 25, 2013

(54) Imaging Feedback of Histotripsy Treatments with Ultrasound Transient Elastography

(71) Applicants: Charles A. Cain, Ann Arbor, MI (US); Tzu-Yin Wang, Taichung City (TW)

(72) Inventors: Charles A. Cain, Ann Arbor, MI (US); Tzu-Yin Wang, Taichung City (TW)

(21) Appl. No.: 13/648,965

(22) Filed: Oct. 10, 2012

Related U.S. Application Data
(60) Provisional application No. 61/545,466, filed on Oct. 10, 2011.

(51) Int. Cl. A61N 7/00

(52) U.S. Cl. CPC A61N 7/00
USPC 601/2

(57) ABSTRACT

Methods and devices for imaging tissue elasticity change as a tool to provide feedback for histotripsy treatments. Tissue lesion elasticity was measured with ultrasound shear wave elastography, where a quasi-planar shear wave was induced by acoustic radiation force generated by the therapeutic array, and tracked with ultrasound imaging at 3000 frames per second. Based on the shear wave velocity calculated from the sequentially captured frames, the Young's modulus in the lesion area was reconstructed. Results showed that the lesions were clearly identified on the elasticity images as an area with decreased elasticity. Lesions produced by histotripsy can be detected with high sensitivity using shear wave elastography. Decrease in the tissue elasticity corresponds well with the morphological and histological change.

### CROSS REFERENCE TO RELATED APPLICATIONS

This application claims the benefit under 35 U.S.C. 119 of U.S. Provisional Patent Application No. 61/545,466, filed Oct. 10, 2011, titled “Imaging Feedback of Histotripsy Treatments with Ultrasound Transient Elastography”, which application is incorporated herein by reference.

### US GOVERNMENT RIGHTS

This invention was made with government support under NIH RO1 CA134579 awarded by the National Institutes of Health. The government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE DISCLOSURE

The present disclosure generally relates to pulsed cavitational ultrasound treatment of tissue. More specifically, the present disclosure relates to improving methods of performing histotripsy therapy by improving imaging feedback with ultrasound elastography.

### BACKGROUND

Histotripsy is a cavitation-based tissue ablation therapy that mechanically fractionates soft tissues using high intensity extremely short ultrasound pulses. During the treatments, the tissues progressively transform from soft solids to fluid-like homogenate. This technique has been shown to successfully fractionate target tissues with high precision in many in vivo models, demonstrating its potential to become a useful therapy tool for noninvasive tissue removal.

Various means are available to assess, in real time, the progression of a histotripsy surgical procedure. The most common method is to wait until the treated volume appears “hypoechoic” (dark) in a standard B-mode ultrasound image. This happens when the tissue is fractionated to a level where surviving fragments are too small to produce significant ultrasound backscatter. This so called “speckle reduction” method is effective but not very sensitive showing reduced speckle amplitude only after very severe mechanical fractionation wherein even the most resilient tissue or cellular organelles are mostly reduced to a liquefied homogenate. Waiting until this level of tissue “reduction” gives a very wide safety margin at the expense of treatment time. For example, if some number of histotripsy pulses destroys all cellular membranes is a treatment volume, no cell will remain viable yet sufficient tissue and cellular elements would survive to continue to support a high level of ultrasound backscatter and therefore, no speckle amplitude reduction in the ultrasound image. The tissue would have to be further reduced, much beyond the effective dose, before sufficient speckle reduction would register to allow termination of treatment of the assessed volume.

Image-based feedback information about the treatment efficacy during and after the treatments is important for a non-invasive therapy like histotripsy. A previous study has shown that ultrasound backscatter reduced in a volume treated by histotripsy, likely because the scattering structures are fractionated to small debris that no longer scatters ultrasound effectively. A following study further demonstrated that quantitative measurement of the backscatter intensity change can be used to provide feedback metric for the degree of tissue fractionation. The backscatter measurement, however, is not sensitive enough to detect the tissue damage at an early stage of the treatments. More sensitive measurement can be achieved with magnetic resonance (MR) T2 weighted imaging. The major drawback of MR is the high cost and the requirement of MR-compatible ultrasound therapy system to provide feedback during the treatments.

### SUMMARY

The present invention relates to methods, procedures, materials, and devices based on elastography that produce quantitative or qualitative feedback allowing assessment of the level of tissue fractionation produced by histotripsy. Elastography measures, and then presents, either in image format, or with numbers, spatial and temporal distributions of the elastic properties of a material, usually tissue (in the context of this disclosure). Measurement of elastic parameter changes due to treatment could involve any assessment modality or imaging approach including but not limited to: ultrasound MRI, or CT, or could involve direct mechanical measurements not involving imaging modalities.

Histotripsy periodically generates, in response to intense ultrasound pulses, energetic bubble clouds that mechanically fractionate tissue, usually within a defined focal volume. The best existing feedback method, speckle reduction, produces hypoechoic (“dark”) zones in ultrasound images as the tissue is fractionated into particles too small to produce significant ultrasonic backscatter. While effective, the tissue fractionation producing visible hypoechoic images is well beyond what is necessary to assure all cells in the treated volume are “dead” (reproductively nonviable). Therefore, in order to reduce the histotripsy “dose”, and subsequent treatment time, a more sensitive method is needed to assess lower degrees of tissue fractionation. Elastography is such a method allowing a virtually continuous measurement of a monotonic decrease in elastic properties (e.g., Young's Modulus or the Shear Modulus) with increasing histotripsy dose. Histotripsy “dose” can be usefully thought of as the number of high intensity focused ultrasound pulses, with a fixed set of pulse parameters, delivered to a fixed focal or overall treatment volume).

The present invention provides methods and devices to allow real time assessment of a therapeutic procedure involving cavitation therapy (histotripsy) to obtain, e.g., a dose sensitive quantitative measure (characterization parameter or set of parameters) from the treated volume that predicts some clinical outcome. Thus, by careful experimental comparison of treated tissue histology with the characterization elastic parameters (CEPs), one might estimate, during or after treatment) the level of tissue damage. This level of histologically verified damage could be correlated to clinical outcomes. By this method, a surgeon would be able to do a treatment and get an immediate noninvasive (no biopsy) assessment of whether or not the target volume was sufficiently fractionated based on a spatial distribution, presented, most likely in image format, of the measured tissue CEPs.

Elastographic methods have been developed for both MRI and ultrasonic imaging modalities. In its most usual form, elastography assesses the elastic properties by launching a shear (transverse) wave that propagates at very low velocity (10s of meters per second as opposed to longitudinal waves that propagate at 1000s of meters per second). The slow propagation allow specially modified ultrasound scanners (higher frame rate) and MRI machines (special very fast imaging sequences) to measure actual shear wave velocity in a whole image plane (ultrasound) or volume (MRI). Since the desired elastic parameters (Young's modulus and/or Shear modulus) are proportional to shear wave velocity, one can get a spatial distribution of these parameters.

Moreover, as tissue is fractionated, the long range elastic connections are successively broken reducing the ability of tissue to “rebound” from a mechanical disturbance, usually in the form of a localized “push.” Increasing fractionation makes the tissue more liquid, reduces monotonically the shear wave velocity (as well as the elastic moduli) until, in a highly homogenized form, no elastic long range connections exist to allow elastic rebound wherein no shear wave can propagate at all. Videos of shear wave propagation, from which the elastic moduli are calculated, clearly show shear waves propagating from the initiating “push” to a homogenized zone and rapidly attenuating in the homogenized zone but continuing to propagate around the non-liquefied surrounding volume.

As shown below, shear wave propagation begins to be affected with only a small histotripsy dose (number of pulses) and continues to show changes in the elastic properties of the treated volume up to very complete homogenization where speckle reduction begins to show the treated zone as much darker (hypoechoic) that surrounding untreated zones. Thus, elastography is much more sensitive than speckle reduction but gives similar image based results useful as a quantitative or qualitative feedback for a given treated zone.

It should be noted that the method of “pushing” the tissue is important to a successful elastographic outcome, and that acoustic pulses, based on radiation pressure, have been successfully used (well described in prior art) for pushing tissues noninvasively at depth.

In therapeutic histotripsy procedures cavitation is not only allowed but is a necessary part of the procedure. Thus, patentable pushing schemes above the cavitation threshold involving interaction of the pushing pulses with the generated bubble clouds (large enhancement of the pushing forces) are possible and will be the subject of further elaboration in what follows.

Pushing embodiments for specific treatment systems are possible that may greatly enhance the feedback capabilities of elastography. For example, in the treatment of BPH or prostate cancer, an ultrasound trans-rectal imaging probe is within centimeters of treatment volume allowing, with appropriate imaging systems, very high quality elastographic images. Current elastography imaging systems begin to degrade in quality where the imaged volume is beyond about 5 cm of depth).

Moreover, the close proximity of the transrectal probe allows several embodiments of a direct transrectal mechanical pushing modality allowing much higher quality elastographic images. Since a modified Foley or other intra-urethra catheter can be placed in the core volume of the prostate, highly localized shear waves can be generated by catheter based pushing allowing, with a nearby trans-rectal ultrasound imaging probe, very high quality elastographic imaging during and after histotripsy treatment of volumes surrounding the “pushing” catheter.

Elastography, whether based on MRI or ultrasound imaging, promises to provide high resolution very sensitive feedback for cavitation or histotripsy therapy. This sensitivity is because of the high sensitivity of shear wave propagation to the fractionation of tissue reducing the ability of tissue to rebound from externally induced displacements (pushes) by whatever means (acoustic radiation force induced displacements or direct displacements by direct mechanical means).

One aspect of the invention is the use of acoustic radiation force pushing at intensities above the cavitation threshold. Current elastographic systems are primarily diagnostic in nature and acoustic pulses above the cavitation threshold are avoided for safety concerns (as mandated by the US Food and Drug Administration). These high intensity pulses generate bubble clouds that effectively reflect the incident ultrasound therefore increasing the rate of change of acoustic momentum, and, therefore, radiation pressure. The combination of much higher allowable intensities and the creation of bubble clouds greatly increases the magnitude of the pushing displacements available for elastographic imaging. This improves resolution and signal to noise ratio and overall image quality at greater target depths compared to pushes from the much weaker pulses available for diagnostic imaging.

Another aspect of this invention is the use of acoustic radiation force pushing outside of the treated zone from two sides. This approach avoids a problem inherent in shear wave imaging of increasingly homogenized treatment volumes. As the treatment progresses, the shear waves cannot propagate very far into the treated zones leaving a “shadow” on the opposite side where no measurement is possible without modifying the approach. The approach discussed below is to push on both sides and to combine the resulting shear wave images (and computed elastic moduli) to get a two-sided complete elastographic image.

Yet another aspect of the invention is the use of acoustic radiation force pushing within a treated volume. Instead of pushing outside the treated zone and watching propagation of shear waves into the treated volume, the pushing is done inside the treated volume and the shear wave that propagates out of that volume is evaluated for parameters indicative of the degree of tissue fractionation. One can watch a single point outside the volume and measure parameter changes due to treatment by pushing inside the volume. This has the huge advantage of obtaining a dose dependent progression of parameter changes at a single point therefore not requiring high frame rate imaging to look at the whole treated volume during shear wave propagation. As outlined below, standard ultrasound imaging systems in the “M-Mode” are capable of making the necessary measurements. Pushing inside the treated volume, instead of outside it, gives a number of measurable parameters that change significantly, at a point outside the treatment volume, as the treatment progresses. In summary, assessment of treatment volume fractionation is possible without high frame rate imaging using off the shelf ultrasound imaging systems.

Some clinical applications may allow very specific useful elastography implementations that are not covered by prior art. In the case of benign prostate hypertrophy (BPH), the target (prostate) is very close to the transrectal imaging probe and is only several millimeters from the rectal wall upon which the imaging probe is likely to rest. Most elastography applications are limited to depths of around 5 cm or less before poor signal to noise ratios limit elastographic image quality. Thus BPH, where the prostate target is always below about 3 cm in depth, is a good target application for elastography feedback. Because of the immediate contact with the rectal wall, the transrectal probe can also accommodate direct mechanical pushing (shear wave excitation) modalities obviating the need for inefficient acoustic pushing approaches. Pushing with a mechanical device on the transrectal probe could greatly enhance shear wave amplitudes enhancing overall image quality, and would allow waveforms, e.g., sinusoidal pushes, that are difficult to obtain by acoustic pushing. This will allow a wider range of elastic parameters to be measured, e.g., the frequency dependence of shear wave velocity that is quite pronounced and somewhat difficult to measure by acoustic impulsive pushing. It should be noted that direct mechanical pushing within the treated volume is also possible using a urethral catheter present in the prostate during treatment, or inserted after treatment to make measurements. In this mode, image free measurements can be made with direct mechanical pushing within the prostate and direct mechanical shear wave detection on the transrectal probe. This may allow a simple excitation-detection feedback scheme giving high quality direct measurements without using any imaging modality and, being an integral part of the therapy device product, a good patent target. In such a scheme, the catheter could be the source of the shear waves (the pusher) or it could be the location of the shear wave detector (with the pusher on the transrectal probe).

In some embodiments, a histotripsy therapy system is provided, comprising an ultrasound therapy transducer configured to deliver histotripsy therapy pulses to tissue, and an ultrasound imaging transducer configured to generate shear waves and form elastography images from the shear waves, the ultrasound imaging transducer also being configured to detect varying degrees of tissue homogenization in the tissue resulting from the delivered histotripsy therapy pulses.

In one embodiment, the ultrasound therapy transducer is configured to deliver histotripsy therapy pulses having a peak negative pressure >10 MPa, a duration <50 μs, and a duty cycle <1%.

In another embodiment, the combination of the ultrasound therapy transducer and the ultrasound imaging transducer generates cavitation bubble clouds that increase the magnitude of pushing displacements available for elastography imaging to create higher resolution and improved image quality at greater target depths.

In some embodiments, the ultrasound therapy transducer and the ultrasound imaging transducer are aligned with a treatment zone of the tissue to apply an acoustic radiation force pushing from outside of the treatment zone from two sides.

In another embodiment, the ultrasound therapy transducer and the ultrasound imaging transducer are aligned with a treatment zone of the tissue to apply an acoustic radiation force pushing from inside of the treatment zone.

In some embodiments, the ultrasound imaging transducer comprises a transrectal imaging transducer.

In other embodiments, the ultrasound imaging transducer comprises a urethral catheter imaging transducer.

In one embodiment, a histotripsy therapy system is provided comprising an ultrasound therapy transducer configured to deliver histotripsy therapy pulses to tissue, the ultrasound therapy transducer also being configured to generate shear waves, and an ultrasound imaging transducer configured to form elastography images from the shear waves and also being configured to detect varying degrees of tissue homogenization in the tissue resulting from the delivered histotripsy therapy pulses.

In some embodiments, the ultrasound therapy transducer is configured to deliver histotripsy therapy pulses having a peak negative pressure >10 MPa, a duration <50 μs, and a duty cycle <1%.

In other embodiments, the combination of the ultrasound therapy transducer and the ultrasound imaging transducer generates cavitation bubble clouds that increase the magnitude of pushing displacements available for elastography imaging to create higher resolution and improved image quality at greater target depths.

In another embodiment, the ultrasound therapy transducer and the ultrasound imaging transducer are aligned with a treatment zone of the tissue to apply an acoustic radiation force pushing from outside of the treatment zone from two sides.

In one embodiment, the ultrasound therapy transducer and the ultrasound imaging transducer are aligned with a treatment zone of the tissue to apply an acoustic radiation force pushing from inside of the treatment zone.

In another embodiment, the ultrasound imaging transducer comprises a transrectal imaging transducer.

In some embodiments, the ultrasound imaging transducer comprises a urethral catheter imaging transducer.

A method of performing histotripsy therapy is provided, comprising delivering histotripsy therapy pulses from a histotripsy therapy transducer to generate acoustic cavitation in a volume of human tissue, generating and directing shear waves towards the volume of human tissue, forming elastography images from the shear waves with an ultrasound imaging transducer, and detecting varying degrees of tissue homogenization in the tissue volume resulting from the delivered histotripsy therapy pulses.

In some embodiments, the generating step further comprises generating and directing the shear waves with the ultrasound imaging transducer.

In other embodiments, the generating step further comprises generating and directing the shear waves with the histotripsy therapy transducer.

In some embodiments, the delivering histotripsy therapy pulses step comprises delivering ultrasound pulses having a peak negative pressure >10 MPa, a duration <50 μs, and a duty cycle <1%.

In some embodiments, the method further comprises applying an acoustic radiation force pushing from outside of the volume of human tissue from two sides.

In other embodiments, the method further comprises applying an acoustic radiation force pushing from inside the volume of human tissue.

In additional embodiments, the method further comprises inserting the ultrasound imaging transducer into a rectum of a patient.

In one embodiment, the method further comprises inserting the ultrasound imaging transducer into a urethra of a patient.

In some embodiments, the generating and directing shear waves step is performed with a catheter inserted into a urethra of a patient.

A histotripsy therapy system is provided, comprising an ultrasound therapy transducer configured to deliver histotripsy therapy pulses to tissue, a shear wave device configured to generate shear waves, and an ultrasound imaging transducer configured to form elastography images from the shear waves and also being configured to detect varying degrees of tissue homogenization in the tissue resulting from the delivered histotripsy therapy pulses.

In some embodiments, the ultrasound therapy transducer is configured to deliver histotripsy therapy pulses having a peak negative pressure >10 MPa, a duration <50 μs, and a duty cycle <1%.

### BRIEF DESCRIPTION OF THE DRAWINGS

FIG. 1 shows an experimental setup comprising a therapeutic histotripsy array, an imaging probe, and a tissue phantom.

FIG. 2 shows a pressure waveform of a 3-cycle, 750-kHz therapy pulse measured in the free field.

FIG. 3 illustrates a timeline of shear wave imaging.

FIGS. 4(a)-(c) show different images acquired at different times after the shear wave generation in tissue phantoms.

FIG. 5 shows (a) B-mode and (b) elasticity images of a representative lesion produced in tissue phantoms.

FIG. 6 illustrates Young's modulus of lesions produced in tissue phantoms.

FIGS. 7(a)-(c) show displacement images acquired at different times after the shear wave generation in ex vivo kidneys.

FIG. 8 shows (a) B-mode and (b) elasticity images of a representative lesion produced in tissue phantoms.

FIG. 9 shows a B-mode image, elasticity map, and gross morphology of a representative lesion produced in the kidneys.

FIG. 10 illustrates Young's modulus of lesions produced in ex vivo kidneys.

FIG. 11 shows histological sections of lesions produced in the ex vivo kidneys.

FIG. 12 shows that the percentage of structurally intact cell nuclei remaining in the treated area decreased exponentially with increasing numbers of pulses.

FIG. 13 shows the correlation between the percentage of remaining structurally intact nuclei and the Young's modulus.

FIGS. 14(a)-(e) show image compounding applied on a lesion produced in the tissue phantoms.

### DETAILED DESCRIPTION

Ultrasound elastography (or elasticity imaging) may be a cost-effective imaging alternative that detects the histotripsy lesions with high sensitivity. This imaging modality measures the tissue elasticity with a spatial resolution comparable to conventional B-mode ultrasound imaging. The general approach for elastography includes: application of stress, estimation of stress-induced strain, and reconstruction of tissue elasticity from the stress-strain relations. The stress can be applied with static or sinusoidal mechanical compression directly exerted on the tissues with mechanical compressors. However, the mechanical compression limits the applicable imaging range to superficial tissues due to the difficulty of coupling the force to deep-lying tissues. Furthermore, artifacts may arise from incomplete knowledge of the boundary conditions. As such, an alternative approach has been developed to remotely apply the stress in the deep-lying tissues with acoustic radiation force. The acoustic radiation force is generated in the tissues along the propagation path of ultrasound by the momentum transfer from the acoustic wave to the medium via absorption and/or reflection of ultrasound. A short duration (˜ms) of focused ultrasound can induce an impulsive ‘push’ in the focal region, which subsequently launches transient shear waves propagating laterally away from the focal region. Because the velocity and attenuation of the shear waves are directly related to the elasticity and viscosity of the tissues, the elasticity can be derived from spatial-temporal recording of the shear waves by direct inversion of the Helmhotz equation, or estimation of the local propagation velocity.

The elastography can provide higher specificity and sensitivity for disease diagnosis due to the high elasticity contrast between diseased and normal tissues. The elastography has been successful in diagnosis of breast cancer, liver cirrhosis, renal disease, and in detection of thermal lesions which are stiffened due to protein denature. In contrast to thermal therapy which produces stiffened tissues, the histotripsy treatments result in soft homogenized tissues in the treated volume. Such tissue transformation may be detected with high sensitivity using elastography due to the potentially high contrast between the elasticity between normal vs. treated tissues.

Elasticity of the treated tissues gradually decreases as the tissues are progressively fractionated with increasing numbers of therapy pulses. The feasibility of imaging the elasticity change using shear wave elastography was determined, where shear waves were induced by an impulsive ‘push’ generated by the therapeutic array transducer and tracked with high frame rate ultrasound imaging. Next, quantitative correlation between the tissue elasticity (i.e., Young's modulus) and the degree of tissue fractionation was studied. The quantitative correlation between the lesion elasticity and the degree of tissue fractionation, if established, provides a basis for predicting the treatment outcomes with tissue elasticity.

Materials and Methods

Sample Preparation

Experiments were performed on agar-graphite tissue mimicking phantoms and ex vivo kidneys. To prepare the agar-graphite tissue mimicking phantoms, agar powder was mixed in deionized water at 0.8% w/v concentration. The mixture was heated in a microwave oven until the agar completely melted in the solution. Next, the graphite powder was mixed in the agar solution at 3% w/v concentration. To ensure homogeneous distribution of graphite in the phantom, the agar-graphite solution was stirred with a magnetic stirrer until it was fully solidified. Although the phantom prepared with this protocol appears to be macroscopically homogeneous, it contains 100-200 μm graphite aggregates under microscopic examination. These aggregates can be fractionated into very fine (<30 μm) debris after the histotripsy treatments. Morphologically, the treated volume also transforms into paste-like homogenate which never re-solidifies to gel (a sign of mechanical rather than thermal damage).

Freshly excised porcine kidneys were obtained from a local meat processing facility. The kidneys were placed in normal saline at 4° C. with the capsule removed. All samples were used within 48 hours of harvest. Prior to experimentation, the kidneys were submerged in degassed (20-30% of normal saturation determined by pO2) saline at room temperature for ˜1 hour. The kidneys were then dissected across the long axis, resulting in two ˜6 cm thick sections with ˜6 cm×˜5 cm cut surface. The kidney sections were embedded in 0.8% agar gel prepared with normal saline in a polycarbonate holder.

Experimental Set-Up

A 750-kHz therapeutic array transducer was used to generate both the therapy pulses for histotripsy and the ‘push’ pulses for shear wave imaging. The transducer has a geometric focal length of 12 cm, with an aperture size of 15 cm and a center hole of 5.9 cm in diameter. The array consists of 9 5-mm wide concentric rings, each dissected into two half-ring elements. The amplitude and phase of the driving signal to each element can be individually controlled by a custom-built array driving system, allowing for electronic steering in the axial direction and F/# control of the transmitted ultrasound.

A 5-MHz 128-element linear array imaging probe connected with a research ultrasound imaging system was used to collect the image data. The imaging system can simultaneously transmit imaging pulses to 128 channels and receive echo signals from 64 channels a time. The driving voltage, center frequency, duty cycle, pulse length, and delay are adjustable. The received signals are sampled with 12-bit analog-to-digital converters at a programmable rate up to 60 MHz.

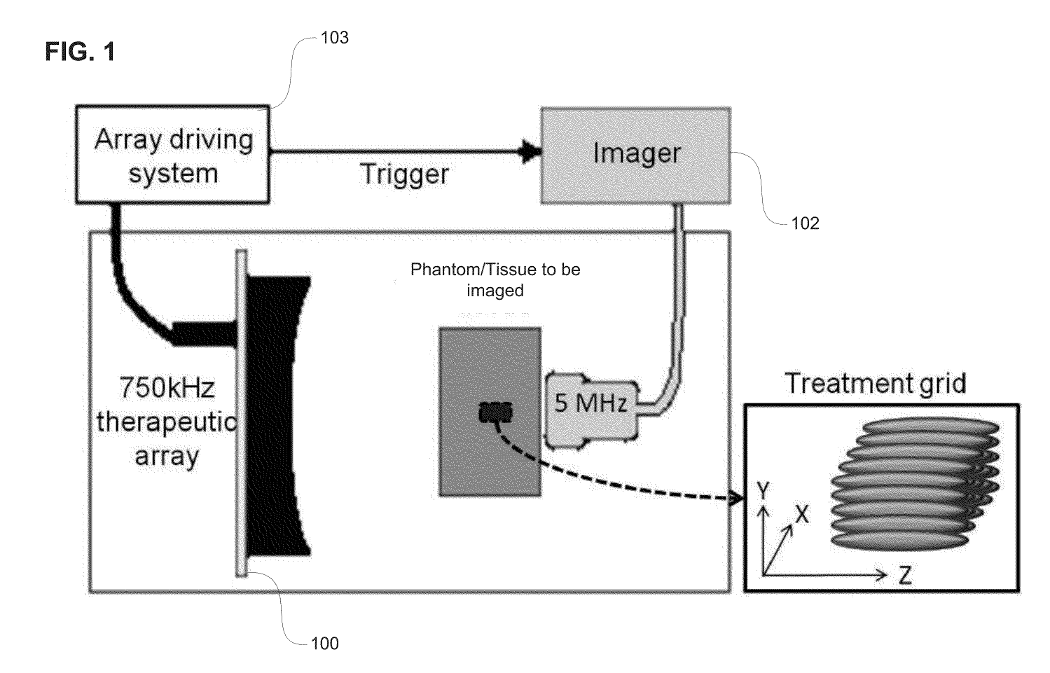

The experimental apparatus is shown in FIG. 1. A therapeutic transducer configured to perform histotripsy therapy on a phantom or tissue to be imaged can be coupled to a histotripsy array driving system 103 and mounted to a manual 3-axis positioning system. Imaging probe 102 can be mounted opposite to the therapeutic array. The beam axis of the therapeutic transducer can be aligned to the imaging plane by the following approach. First, a bubble cloud can be induced in the water with brief excitation of the therapeutic transducer. The therapeutic transducer can be placed so that the bubble cloud appeared with highest backscatter amplitude and largest spatial extent on the ultrasound images. After the alignment, the phantom or tissue can be mounted to a motorized 3-axis positioning system and submerged in the tank approximately at the geometric focus of the therapeutic transducer. For the tissue experiments, the kidneys were placed with the long axis parallel to the ultrasound beam axis. This orientation allows the target volume to be imaged and treated in the cortex area without interference from the collecting system.

Histotripsy Treatments

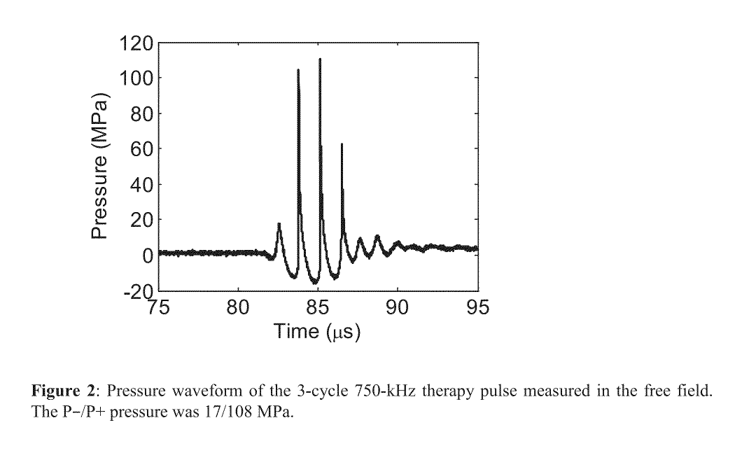

Histotripsy therapy pulses of 3 cycles in duration were delivered at 50 Hz pulse repetition frequency (PRF) by the therapeutic array transducer. All elements on the array were driven in-phase with equal amplitude, resulting in an F/# 0.8 focal configuration. The pressure field was calibrated with a custom-built fiber optic probe hydrophone (FOPH) with an active element of 100 μm in diameter. The peak negative (P−) and peak positive (P+) pressures were measured −17 and 108 MPa, respectively (FIG. 2). Due to attenuation of ultrasound in the agar-graphite phantoms (0.1 dB/cm/MHz) and in the kidneys (1 dB/cm/MHz), the P− pressures at the treatment location were likely ˜16.5 MPa in the phantoms, and ˜13 MPa in the kidneys. The P+ pressures were likely decayed more significantly due to nonlinear absorption. The −6-db beamwidths were measured at a reduced P−/P+ pressure of −11/58 MPa. The lateral/axial −6-db beamwidth were 2.6/17.8 mm on the P− pressure profile, and 1.2/7.3 mm on the P+ pressure profile. The beamwidths at higher pressures could not be successfully measured because the cavitation easily occurred and damaged the fiber tip during the pressure profile scan.

Lesions of approximately 7 mm×7 mm×14 mm were produced by mechanically scanning the therapy focus in a 5×5 grid with 1 mm spacing on the focal plane (FIG. 1). To produce different degrees of tissue fractionation, lesions were produced with treatment doses of 0, 100, 200, 300, 500, 1000, 1500, and 2000 therapy pulses per treatment location on the grid. A total of 72 treatments, 9 for each dose, were performed on the agar-graphite tissue phantoms. A total of 64 treatments, 8 for each dose, were performed on the ex vivo kidneys. After the treatments, the samples were moved 15 mm laterally so that the elasticity imaging was performed with the shear waves excited from outside and propagating across the lesions.

Shear Wave Elastography

The shear wave elastography approach used in this study is similar to those described in the literature. To generate a quasi-planar shear wave, three ultrasound ‘pushing’ beams were sequentially fired by the therapeutic array transducer focused at z=+5, 0, and −5 mm with respect to the geometric focus (i.e., z=120 mm). A 2-μs delay was inserted between push beams to allow the array driving system to update the new focal location. For each pushing beams, the outer 5 rings were shut off, resulting in a F/#−1.2 focal configuration. The pulse parameters and the measured pressure fields of the three beams were summarized in Table 1.

TABLE 1
Pulse parameters for acoustic radiation force generation under free field conditions.

| | | Push beam 1 | | Push beam 2 | | Push beam 3 | |
|---|---|---|---|---|---|---|---|
| Focal location | | z = 125 | mm | z = 120 | mm | z = 115 | mm |
| −6 db beamwidths on P− pressure | | 3.4/34.7 | mm | 3.6/31.5 | mm | 3.1/29.7 | mm |
| profile (lateral/axial) | | | | | | | |
| −6 db beamwidths on P+ pressure | | 1.8/23.0 | mm | 1.6/20.0 | mm | 1.6/17.8 | mm |
| profile (lateral/axial) | | | | | | | |
| P−/P+ Pressure* | phantom | −5/14 | MPa | −5/15 | MPa | −5/16 | MPa |
| | experiments | | | | | | |
| | ex vivo tissue | −6/30 | MPa | −6/34 | MPa | −7/36 | MPa |
| | experiments | | | | | | |
| Pulse duration | phantom | 133 | μs | 133 | μs | 133 | μs |
| | experiments | | | | | | |
| | ex vivo tissue | 200 | μs | 200 | μs | 200 | μs |
| | experiments | | | | | | |
| I_{SPPA}** | phantom | 1.3 | kW/cm² | 1.5 | kW/cm² | 1.6 | kW/cm² |
| | experiments | | | | | | |
| | ex vivo tissue | 2.3 | kW/cm² | 2.5 | kW/cm² | 2.8 | kW/cm² |
| | experiments | | | | | | |

*The pressures at the treatment locations were expected lower than these measurements due to sound attenuation. Given a mean propagation distance of 3 cm, and attenuation coefficients of 0.1 dB/cm/MHz in the agar-graphite phantoms [49], and 1 dB/cm/MHz in the kidneys [50], the P− pressures were estimated ~5 MPa both in the phantom and the ex vivo experiments.
**I_{SPPA}: Spatial peak pulse average intensity.

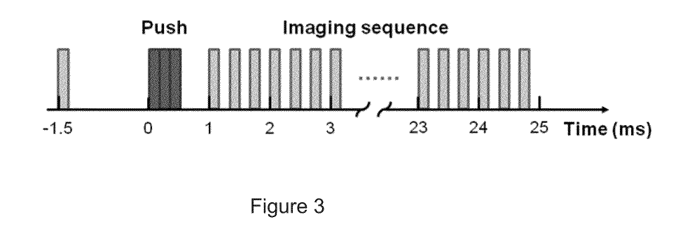

To track the propagation of the shear waves at a high frame rate, ultrasound plane wave imaging was performed. The imaging system generated a plane wave to illuminate the entire imaging field by simultaneously transmitting the imaging pulse from all elements on the imaging probe. The system then started recording the backscatter signals immediately after the plane wave was transmitted. As the system can only receives signals from 64 channels a time, 2 successive transmit-receive cycles, each lasting for 77 μs, were conducted to collect the backscatter signals from the 38 mm×38 mm field of view. A total of 73 frames were captured: one reference frame acquired 1 ms before the pushing beams, and 72 frames acquired at a rate of 3000 frames per second from 1 ms after the pushing beams (FIG. 3). The total imaging duration lasted for 24 ms.

The channel data were collected and processed off-line. Conventional delay-and-sum beamforming with a F/# 1.5 dynamic receive focusing was applied to produce the beamformed radio-frequency (RF) images. The 1-D correlation based speckle tracking algorithm was then applied on the beamformed RF data to estimate the local tissue displacement. The RF data were segmented into 1.5 mm regions with 75% overlap along the axial direction. The cross correlation function of the RF segments from consecutive frames was calculated. The position of the maximum of the cross correlation function was obtained by locating the phase zero crossing around the maximum magnitude of the function. This position determined the tissue displacement between the consecutive frames.

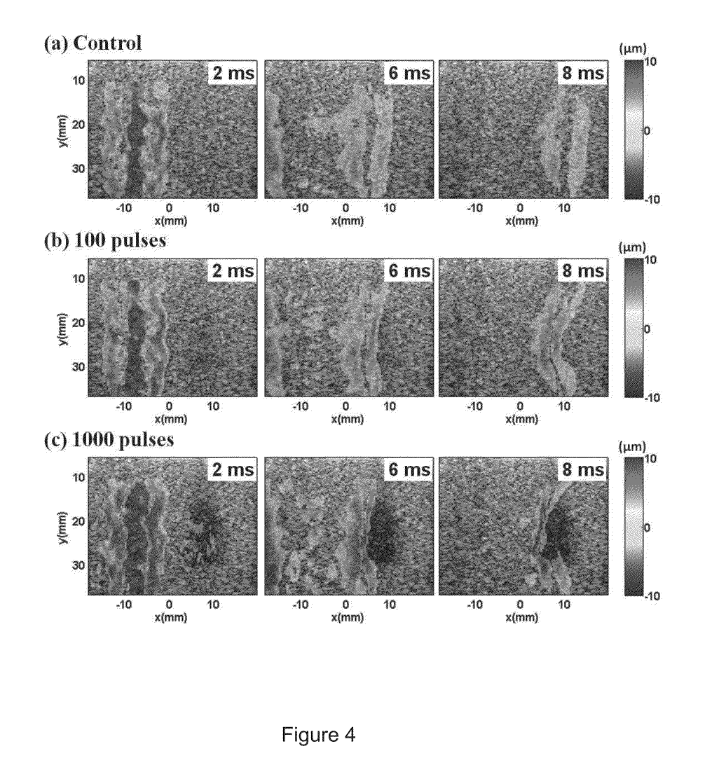

The above processing produced a series of spatial-temporal displacement images (e.g., FIG. 4) which allows the estimation of the shear wave propagation velocity in each local area, (x, z), with a time-of-flight algorithm. For each location (x, z), the temporal displacement profiles at two points across the location, u(x−Δx, z, t) and u(x+Δx, z, t), were extracted from the spatial-temporal displacement images. The propagation time, At, between the two points was obtained from the location of the maximum of the cross correlation function of the two displacement profiles, u(x−Δx, z, t) and u(x+Δx, z, t). The propagation velocity was calculated as vs=2.Δx/Δt. To improve to quality of measurement, an average propagation velocity was calculated from multiple estimates with different spacing, Δx. In this study, the average velocity was calculated from 3 pairs of points with Δx=0.6-0.9 mm. This set the resolution of the final elasticity map to be approximately 1.8 mm. Finally, the Young's modulus was calculated by

E=3μ=3 ρvₛ²

where E is the Young's modulus, μ is shear modulus, and ρ is the density of the medium (assumed 1000 kg/m3 for the phantoms and the tissues).

The imaging process was repeated 3 times for the lesions created in the agar-graphite phantoms, and 9 times for those created in the kidneys. An average was obtained from the repeated measurements. A median Young's modulus was calculated for each lesion in an 8×6 mm region approximately in the center of the lesions.

To validate the elasticity measured with ultrasound imaging, the elasticity of the untreated samples was also measured with a custom-built elastometer. The phantoms or the kidney cortex were cut into 1 cm cubes and placed on an electronic balance. An aluminum rod with 8-mm diameter flat end was brought in contact with the samples. The rod was pressed into the samples step by step with a known step size. The scale reading was recorded to estimate the applied force for each step. A linear fit was applied to the stress-strain curves in the low strain (<10%) regime. The slope of the linear fit was determined as the Young's modulus of the samples.

Histological Examination

After experimentation, the tissues were fixed in 10% neutral buffered formalin and prepared for hematoxylin & eosin (H&E) staining. Histological sections of 4 μm thickness were made at 500 μm intervals through the lesion center with slices oriented in parallel with the ultrasound imaging plane. The sections were examined with a bright field microscope at a 400× magnification.

To evaluate the degree of tissue fractionation, the percentage of structurally intact cell nuclei remaining in the treated area was calculated. The cell nuclei were selected because they are a common indicator of cell or tissue damage. Moreover, they appeared more resistant to histotripsy damage than other cellular components, thus serving as a good indication of histotripsy damage. The calculation follows the process described in our previous publication. In brief, images of five 320 μm ×240 μm regions in the lesion area were captured. The locations of the five regions form a cross pattern with 1.5 mm span in 4 directions and centered approximately at the lesion center. The numbers of structurally intact cell nuclei were counted for the five images. An average of the five counts was obtained and normalized to the average count from an untreated area (control), producing a percentage of remaining structurally intact cell nuclei. This percentage may represent the degree of tissue fractionation caused by histotripsy.

### RESULTS

Experiments on Agar-Graphite Phantoms

FIG. 4 shows the temporal displacement field induced in the phantoms by the shear waves. Displacement of several tens of gm was detected in the push location after the pushing beams were delivered. The shear waves were launched from the push location and propagated outward in the lateral direction. The shear waves appeared to have quasi-planar wavefronts as they propagated across an untreated area (control, FIG. 4a). The shear waves appeared to propagate at a lower velocity in a treated area, leading to a curved wavefront (e.g., FIG. 4b, c). The propagation velocity decreased as the treatment doses increased. At doses higher than 1000 pulses/location, the propagation was so slow that the shear waves could not propagate across the treated area within the 24-ms observation period.

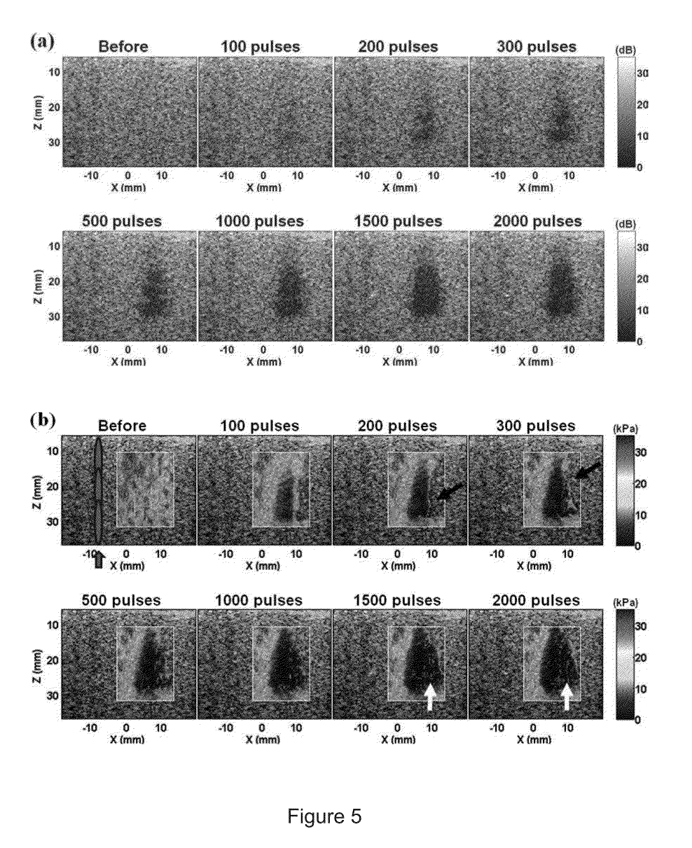

The B-mode and the elasticity images of a representative lesion treated with increasing doses were shown in FIG. 5. As treatment dose increased, the treated area became increasingly hypoechoic on the B-mode images. Correspondingly, significant decrease in the Young's modulus was observed, indicating a softer area produced by histotripsy. The treated area was more easily identified on the elasticity images compared to the B-mode images, particularly at low doses (e.g., 100 pulses per treatment location). The elasticity in the majority of the treated area was successfully measured except for some regions in the far end opposite to the location of shear wave generation. Unsuccessful or noisy measurements were obtained in these regions especially when high treatment doses were applied. This is likely because the shear waves were unable to propagate across a sufficiently fractionated volume.

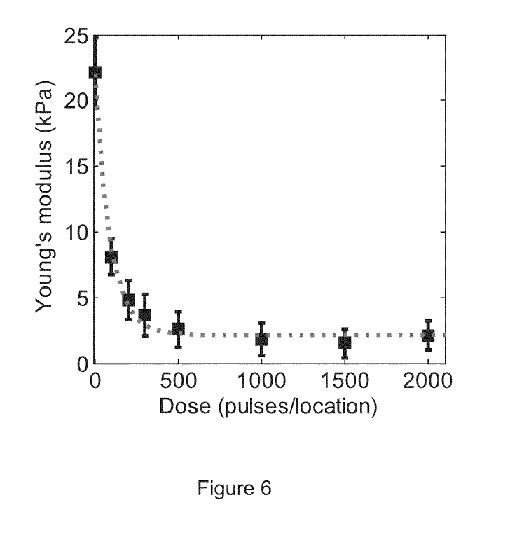

The elasticity, or the Young's modulus, of the treated area decreased exponentially with increasing treatment doses (R2=0.99, FIG. 6). This decrease leveled off when the treatment dose was higher than 500 pulses per treatment location. The Young's modulus of the untreated agarose phantoms measured with shear wave elastography was comparable with that measured with the elastometer, i.e., 26.6±2.6 kPa (N=7).

Experiments on Ex Vivo Kidneys

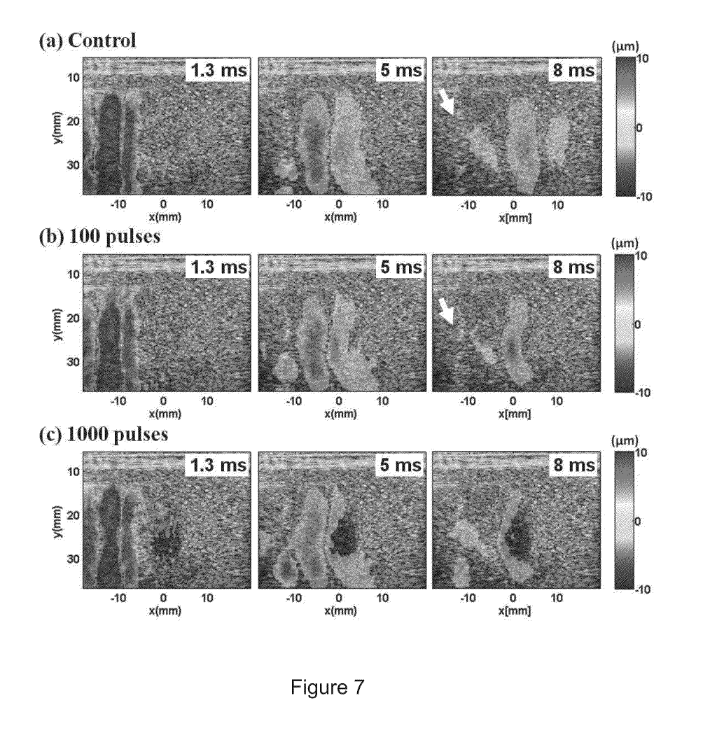

FIG. 7 shows the temporal displacement field induced in the tissues by the shear waves. Similar to the phenomena observed in the phantom study (FIG. 4), shear waves were launched from the push location and propagated laterally. The shear waves slowed down in the treated area, and could not propagate across the lesion when the lesion was created with higher doses (>1000 pulses/location). Comparing the waveforms in the phantoms (FIG. 4), the shear waves in the tissues appeared to spread wider and attenuate faster as they propagate across the medium (FIG. 7). This likely occurs due to the significant dispersion and attenuation of shear waves in the tissues. It is worth noting that cavitation could have been induced at the push location by the pushing beams, e.g., the bright spots indicated by the arrows in FIG. 7.

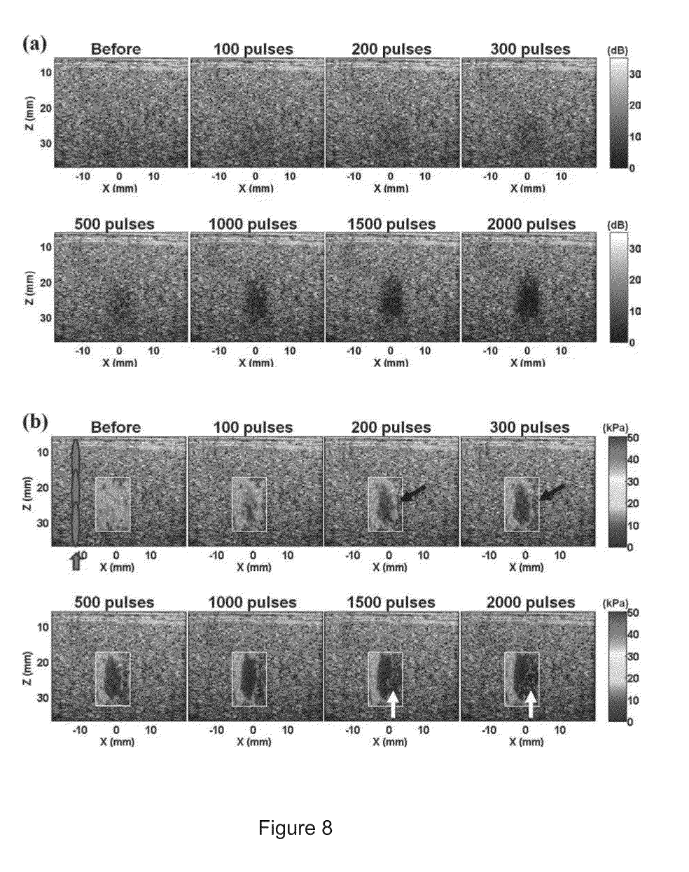

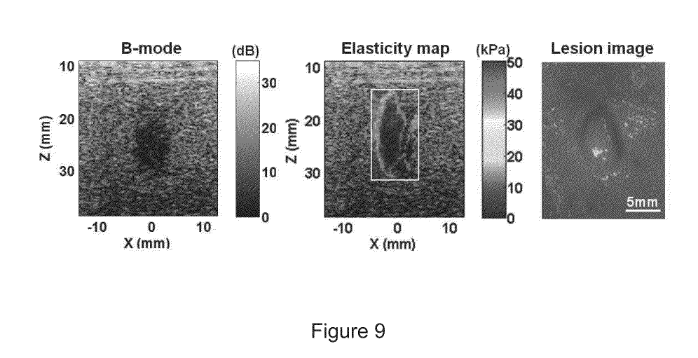

The B-mode and the elasticity images of a representative lesion treated with increasing numbers of pulses were shown in FIG. 8. The treated volume was identified as an increasing hypoechoic area on the B-mode images, and a softer area with decreased Young's modulus on the elasticity images. The treated area was more easily identified on the elasticity images than on the B-mode images at low doses (e.g., <500 pulses per treatment location). Unsuccessful or noisy measurements were obtained in the far end of the lesion opposite to the location of shear wave generation because the shear waves could not propagate well across the fractionated volume. Despite of these interferences, the lesions depicted on the B-mode and elasticity images corresponded well with their morphological appearance (FIG. 9).

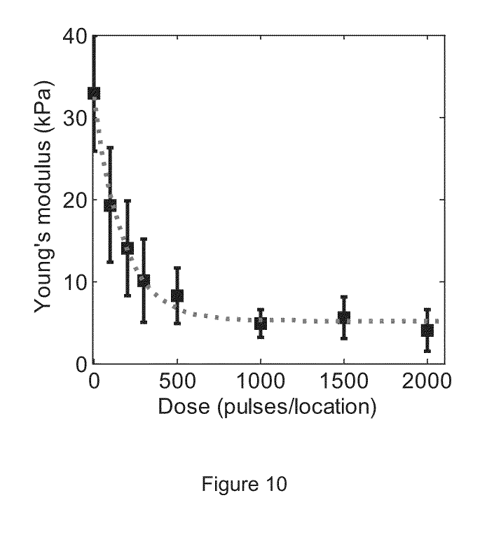

The Young's modulus of the treated area decreased exponentially with increasing treatment doses (R2=0.99, FIG. 10). This decrease leveled off when the treatment dose was higher than 1000 pulses per treatment location. The Young's modulus of the untreated kidney cortex measured with shear wave elastography was comparable with that measured with the elastometer, i.e., 30.2±4.5 kPa (N=8).

Histological Examinations

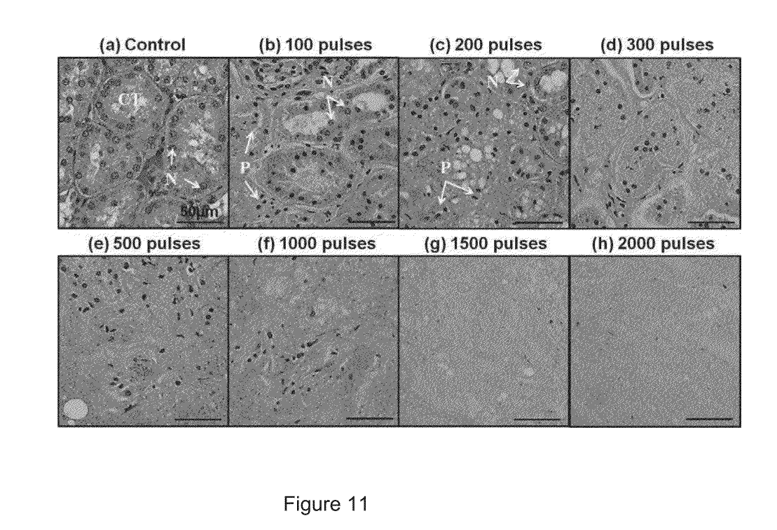

Representative histological sections of the lesions produced with increasing numbers of pulses are shown in FIG. 11. In the control, all tissues structures and cell nuclei appeared structurally intact. In the treated area, damage to both the tissue structures and the cellular components were observed. Increasingly more damage was observed with increasing numbers of therapy pulses. At low doses, a small part of the tissues were damaged while most part remained structurally intact. Some damaged (pyknotic or fragmented) cell nuclei were observed. As the numbers of pulses increased, a larger part of the tissues were damaged and more damaged cell nuclei were found. With a dose higher than 1000 pulses per treatment location, the treated volume appeared to be completely homogenized with no recognizable tissue structures and very few fragments of nuclear material, if present.

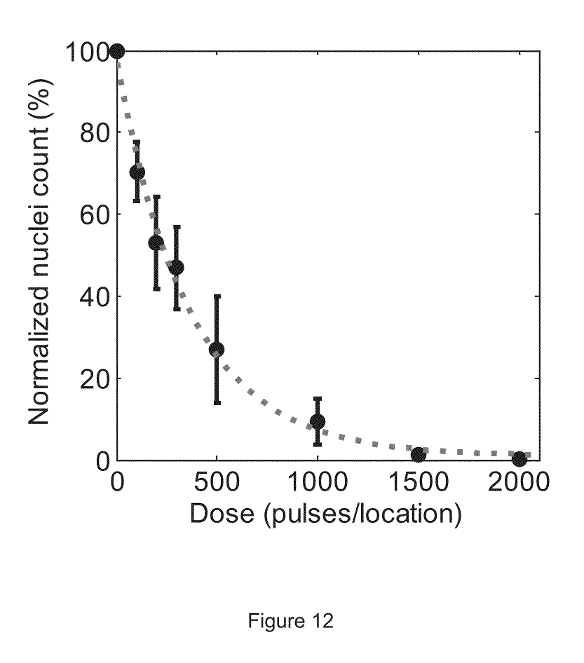

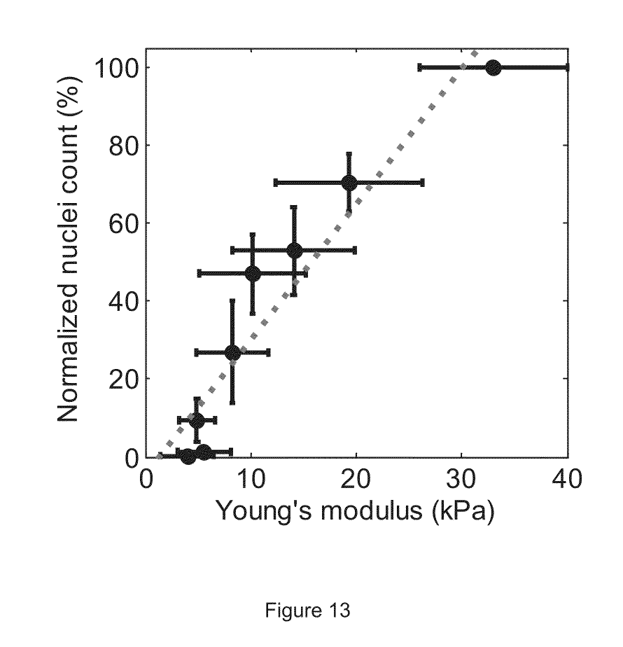

The degree of tissue fractionation as examined with the percentage of remaining intact cell nuclei is plotted against the treatment doses in FIG. 12. The percentage of remaining intact nuclei decreased exponentially with increasing numbers of therapy pulses (R2=0.99). This percentage was further correlated to the Young's modulus of the treated tissues (FIG. 13). A linear correlation was found between the percentage of remaining intact nuclei and the lesion Young's modulus (R2=0.91).

Discussion

The elastography appears to be a more sensitive measurement for detecting tissue fractionation in the early stage of the treatments compared to another imaging feedback metric, backscatter reduction. This is evidenced by the better contrast of the lesion in the elasticity images than in the B-mode images, especially when the lesion was created with low therapy doses. For example, the lesions produced with 100 pulses per treatment location are more easily identified in the elasticity images than in the B-mode images, both in the phantom study (FIG. 5) and in ex vivo tissue study (FIG. 8). The capability of detecting tissue fractionation at the beginning of the treatment is critical for precise targeting, analysis, and optimization of the treatments.

In addition to higher sensitivity, the elastography can compensate for the discrepancy of feedback with backscatter reduction in vivo. Our experience in the in vivo studies has shown that the backscatter reduction is apparent during and within several minutes after the treatments. The backscatter intensity may increase again several minutes later, possibly because the blood coagulated in the treated volume. In this situation, the lesions may still be detectable with elastography since the newly formed blood clot is likely very soft (Young's modulus ˜2 kPa [54]) compared to normal tissues.

A good correlation was found in the present work between the tissue elasticity and the degree of tissue fractionation as indicated by the damage to the cell nuclei. The change in the tissue elasticity, however, could be more directly related to the damage to the connective structures in the tissue (e.g., the extracellular matrix) responsible for tissue elasticity. It is possible that a stronger correlation may be found between the tissue elasticity and the degree of damage to these structures. In spite of such possibility, the correlation found in the present work remains an important result which has significant clinical implications. The correlation suggests that the tissue elasticity can be a very useful indicator for predicting the treatment outcomes produced by histotripsy because the injury to the cell nuclei are highly relevant to tissue damage and many other clinical situations.

The Young's moduli measured with shear wave elastography corresponded well with those measured with the elastomer. These measurements are also comparable to several results reported in the literature. For instance, the Young's modulus of the tissue phantoms measured in this study is comparable to that reported in (20-30 kPa). The Young's modulus of the ex vivo kidney cortex is comparable to results from (20-40 kPa), although higher than results from (7-15 kPa). The higher elasticity obtained in the present work likely occurs for a similar reason discussed by other researchers. The present work estimated the elasticity based on the group velocity of the shear waves induced by an impulse excitation. The waves contain a wide band of frequencies (center frequency =130-220 Hz, −15 dB frequency band =30-500 Hz). In a non-dispersive medium, each frequency should travel at the same velocity as the group velocity. In the tissues, however, the strong dispersion causes higher frequencies to travel at higher velocities. Therefore, the elasticity estimated from the group velocity could be higher than that estimated from the velocity of shear waves induced by a single lower frequency, (e.g., 90 Hz in [60]).

The mechanism of acoustic radiation force generation in the current work may be very different from that in most elastography setups for diagnostic purposes. The acoustic radiation force could be generated via absorption and/or reflection of ultrasound in the tissues. In the setup for diagnostic purpose, the radiation force is generated primarily through absorption. To enhance the absorption within the safety regulations, the radiation force is commonly generated by ultrasound with a higher frequency (5-10 MHz), limited pressure (<5 MPa), and long duration (300-600 μs). In the current work, the acoustic radiation force is generated by ultrasound with similar duration (450-600 μs) but a lower frequency (750 kHz) and a slightly higher pressure (5-6 MPa). The radiation force caused by the absorption could be low compared to other diagnostic setups because the absorption decreases with decreasing frequencies. However, the combination of low frequency, high pressure, and long pulse duration could have increased the likelihood of cavitation. The acoustic radiation force could be generated from the strong reflection from the cavitation bubbles. This is supported by the observation of cavitation bubbles generated in the push location after the push pulses were delivered (FIG. 7). These bubbles produced noticeable damage on the histological sections of the tissues. Therefore, we envision this setup to be applied within the target volume where the push location will be ablated eventually.

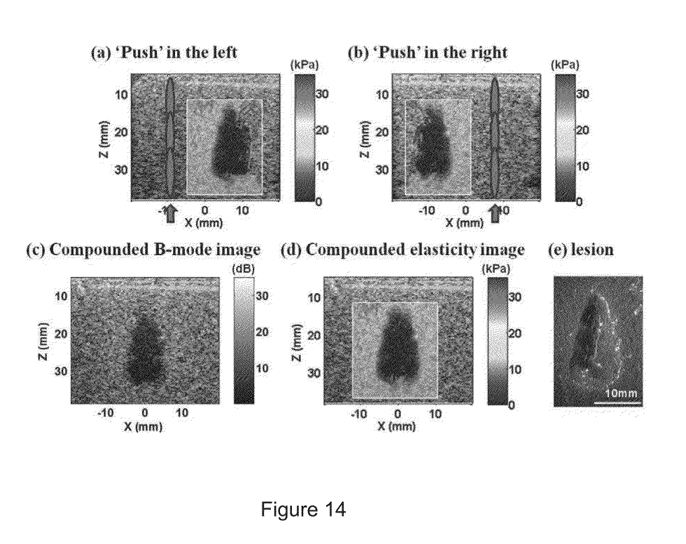

One issue of imaging the histotripsy lesions with shear wave elastography is that the shear waves may not be able to propagate across a sufficiently fractionated volume. This makes it difficult to outline the entire lesion, especially on the far end opposite to the shear wave excitation location. This issue may be addressed with an image compounding technique shown in FIG. 14. Two elasticity images were obtained separately with shear waves excited from the left or right side of the lesion. Using a simple image registration algorithm applied on the B-mode images, the two elasticity images can be combined to provide an elasticity map of the entire lesion.

Challenges may exist when applying the current setup for image feedback in in vivo situations in real time. For example, the computation for an elasticity image currently takes several minutes. This time may be significantly reduced by optimizing the data processing algorithms, allowing for close-to-real-time feedback. Another challenge may arise from significant degradation of the image quality in deep-lying tissues, which could affect the accuracy of tissue displacement tracking. The challenges and solutions will be investigated in the future.

### CONCLUSIONS

Tissues treated with histotripsy become increasingly softer as they are fractionated by increasing numbers of therapy pulses. This tissue transformation process can be detected with high sensitivity using shear wave elastography. The created lesions depicted on the elasticity images correspond well with their morphological appearance. Strong correlation exists between the lesion elasticity and the degree of tissue fractionation as examined by the percentage of remaining structurally intact cell nuclei. Since damage to the cell nuclei is directly related to cell death, tissue injury, and many other clinical outcomes, this correlation provides basis for predicting histotripsy treatment outcomes from tissue elasticity change, allowing for a clinician to determine when sufficient treatment has occurred.

What is claimed is:

1. A histotripsy therapy system, comprising:
an ultrasound therapy transducer configured to deliver histotripsy therapy pulses to tissue; and
an ultrasound imaging transducer configured to generate shear waves and form elastography images from the shear waves, the ultrasound imaging transducer also being configured to detect varying degrees of tissue homogenization in the tissue resulting from the delivered histotripsy therapy pulses.

2. The system of claim 1 wherein the ultrasound therapy transducer is configured to deliver histotripsy therapy pulses having a peak negative pressure >10 MPa, a duration <50 μs, and a duty cycle <1%.

3. The system of claim 1 wherein the combination of the ultrasound therapy transducer and the ultrasound imaging transducer generates cavitation bubble clouds that increase the magnitude of pushing displacements available for elastography imaging to create higher resolution and improved image quality at greater target depths.

4. The system of claim 1 wherein the ultrasound therapy transducer and the ultrasound imaging transducer are aligned with a treatment zone of the tissue to apply an acoustic radiation force pushing from outside of the treatment zone from two sides.

5. The system of claim 1 wherein the ultrasound therapy transducer and the ultrasound imaging transducer are aligned with a treatment zone of the tissue to apply an acoustic radiation force pushing from inside of the treatment zone.

6. The system of claim 1 wherein the ultrasound imaging transducer comprises a transrectal imaging transducer.

7. The system of claim 1 wherein the ultrasound imaging transducer comprises a urethral catheter imaging transducer.

8. A histotripsy therapy system, comprising:
an ultrasound therapy transducer configured to deliver histotripsy therapy pulses to tissue, the ultrasound therapy transducer also being configured to generate shear waves; and
an ultrasound imaging transducer configured to form elastography images from the shear waves and also being configured to detect varying degrees of tissue homogenization in the tissue resulting from the delivered histotripsy therapy pulses.

9. The system of claim 8 wherein the ultrasound therapy transducer is configured to deliver histotripsy therapy pulses having a peak negative pressure >10 MPa, a duration <50 μs, and a duty cycle <1%.

10. The system of claim 8 wherein the combination of the ultrasound therapy transducer and the ultrasound imaging transducer generates cavitation bubble clouds that increase the magnitude of pushing displacements available for elastography imaging to create higher resolution and improved image quality at greater target depths.

11. The system of claim 8 wherein the ultrasound therapy transducer and the ultrasound imaging transducer are aligned with a treatment zone of the tissue to apply an acoustic radiation force pushing from outside of the treatment zone from two sides.

12. The system of claim 8 wherein the ultrasound therapy transducer and the ultrasound imaging transducer are aligned with a treatment zone of the tissue to apply an acoustic radiation force pushing from inside of the treatment zone.

13. The system of claim 8 wherein the ultrasound imaging transducer comprises a transrectal imaging transducer.

14. The system of claim 8 wherein the ultrasound imaging transducer comprises a urethral catheter imaging transducer.

15. A method of performing histotripsy therapy, comprising:
delivering histotripsy therapy pulses from a histotripsy therapy transducer to generate acoustic cavitation in a volume of human tissue;
generating and directing shear waves towards the volume of human tissue;
forming elastography images from the shear waves with an ultrasound imaging transducer; and
detecting varying degrees of tissue homogenization in the tissue volume resulting from the delivered histotripsy therapy pulses.

16. The method of claim 15 wherein the generating step further comprises generating and directing the shear waves with the ultrasound imaging transducer.

17. The method of claim 15 wherein the generating step further comprises generating and directing the shear waves with the histotripsy therapy transducer.

18. The method of claim 15 wherein the delivering histotripsy therapy pulses step comprises delivering ultrasound pulses having a peak negative pressure >10 MPa, a duration <50 μs, and a duty cycle <1%.

19. The method of claim 15 further comprising applying an acoustic radiation force pushing from outside of the volume of human tissue from two sides.

20. The method of claim 15 further comprising applying an acoustic radiation force pushing from inside the volume of human tissue.

21. The method of claim 15 further comprising inserting the ultrasound imaging transducer into a rectum of a patient.

22. The method of claim 15 further comprising inserting the ultrasound imaging transducer into a urethra of a patient.

23. The method of claim 15 wherein the generating and directing shear waves step is performed with a catheter inserted into a urethra of a patient.

24. A histotripsy therapy system, comprising:
an ultrasound therapy transducer configured to deliver histotripsy therapy pulses to tissue;
a shear wave device configured to generate shear waves; and
an ultrasound imaging transducer configured to form elastography images from the shear waves and also being configured to detect varying degrees of tissue homogenization in the tissue resulting from the delivered histotripsy therapy pulses.

25. The system of claim 24 wherein the ultrasound therapy transducer is configured to deliver histotripsy therapy pulses having a peak negative pressure >10 MPa, a duration <50 μs, and a duty cycle <1%.
